(19) United States
(12) Patent Application Publication
(10) Pub. No.: US 2024/0339201 A1
(43) Pub. Date: Oct. 10, 2024

(54) BREAST CANCER-RELATED INFORMATION PROVIDING METHOD USING MAGNETIC RESONANCE IMAGE AND RNA GENETIC INFORMATION

(71) Applicant: KOREA UNIVERSITY RESEARCH AND BUSINESS FOUNDATION, Seoul (KR)

(72) Inventors: Bo Kyoung SEO, Seoul (KR); Ah Young PARK, Gyeonggi-do (KR); Mi-Ryung HAN, Seoul (KR)

(21) Appl. No.: 18/681,982

(22) PCT Filed: Aug. 9, 2022
(86) PCT No.: PCT/KR2022/011883

§ 371 Date: Feb. 7, 2024

(30) Foreign Application Priority Data
Aug. 9, 2021 (KR) 10-2021-0104901

(51) Int. Cl. G16H 30/40; G06T 7/00; G16B 20/00

(52) U.S. Cl. CPC G16H 30/40; G06T 7/0012; G16B 20/00; G06T 2207/10088; G06T 2207/30068; G06T 2207/30096

(57) ABSTRACT

The present invention relates to a method for providing information relating to progression or prognosis of a breast cancer and information for selecting a breast cancer treatment method, the method using a breast cancer MRI. Since genetic information can be predicted by means of a non-invasive MRI when using the method of the present invention, the method may be used to provide information relating to progression or prognosis of a breast cancer or information for selecting a breast cancer treatment method.

### TECHNICAL FIELD

This invention was carried out under the support of the Ministry of Science and ICT of the Republic of Korea, with the project number NRF-2021R1A2C1010565. The project management professional organization is the National Research Foundation of Korea. The research project title is “Construction of a Radiomics Platform for Predicting Breast Cancer Prognosis Genes and Microenvironment Using Artificial Intelligence,” under the program “Basic Research Project for Mid-Career Researchers.” The lead institution is Korea University, and the research period is from Mar. 1, 2021, to Feb. 29, 2024.

Additionally, this invention was conducted with the support of the Ministry of Science and ICT of the Republic of Korea, under the project number 2020R1C1C1012288. The project management professional organization is the National Research Foundation of Korea. The research project title is “Establishment of a Precision Medicine Basis for Women's Cancer Using Integrated Big Data Analysis of Imaging Genomics,” under the program “Basic Research Project in Science and Engineering (Support for Young Researchers).” The lead institution is the Industrial-Academic Cooperation Foundation of Incheon National University, and the research period is from Mar. 1, 2020, to May 28, 2025.

Furthermore, this invention was carried out with the support of the Ministry of Science and ICT of the Republic of Korea, under the project number 2020R1G1A1102372. The project management professional organization is the National Research Foundation of Korea. The research project title is “The Role of Conventional Dynamic Contrast-Enhanced MRI and Ultrafast MRI in Predicting the Response to Preoperative Chemotherapy in Breast Cancer: Diagnostic Ability and Imaging-Pathology Correlation Using Microvascular Density,” under the program “Basic Research Project in Science and Engineering (First Research in Life).” The lead institution is the Industrial-Academic Cooperation Foundation of CHA University, and the research period is from Sep. 1, 2020, to Feb. 28, 2023.

This patent application claims priority to and the benefit of Korean Patent Application No. 10-2021-0104901 filed with the Korean Intellectual Property Office on Aug. 9, 2021, the disclosure of which is incorporated herein by reference.

The present disclosure relates to a method for providing information about the progression or prognosis of breast cancer and information for selecting methods of treatment, using MRI images of breast cancer.

### BACKGROUND ART

The MRI characteristics of breast cancer can be attributed to various genetic changes. However, there is hardly any prospective research on the correlation between multiparametric MRI features and total RNA sequencing data in breast cancer.

Magnetic Resonance Imaging (MRI) is the most sensitive imaging technique with good specificity for diagnosing and assessing the treatment response of breast cancer through multiparametric evaluation. The multiparametric MRI assessment includes the qualitative evaluation of tumor morphology using BI-RADS (Breast Imaging-Reporting and Data System) and the quantitative evaluation of tumor angiogenesis and heterogeneity using computational analysis of time-enhancement curves, perfusion features, and texture features.

Tumor angiogenesis is essential for the progression of breast cancer, and the degree of angiogenesis using MRI can be evaluated through time-intensity curves using commercially available CAD (Computer-Aided Diagnosis) systems. Recent studies have reported that high kinetic heterogeneity and peak enhancement in CAD are associated with poor distant metastasis-free survival in breast cancer. Tumor heterogeneity represents histological complexity including cell density, necrosis, or extracellular matrix and can be measured using texture analysis, which refers to mathematical methods assessing the intensity and position of pixel gray levels. Recent studies have shown that high entropy in T2-weighted images indicating tumor heterogeneity is associated with poor recurrence-free survival. Meanwhile, tumor angiogenesis and heterogeneity can be affected by various genetic mutations.

Radiogenomic investigation in breast cancer can provide imaging biomarkers that help in selecting the optimal treatment and predicting prognosis more accurately by providing a better understanding of tumor characteristics at the genetic level. There have been several retrospective radiogenomic analyses that have correlated features of breast cancer MRI with genetic changes. They have revealed that tumor size, lesion type, shape, or heterogeneous enhancement in contrast-enhanced T1-weighted MRI are correlated with genetic changes related to the cell cycle, recurrence, or the tumor microenvironment. However, there are hardly any prospective studies that have linked clinically accessible multiparametric MRI features with total RNA sequencing data.

### DISCLOSURE OF INVENTION

### Technical Problem

The present inventors have endeavored in previous research efforts to develop clinical outcomes and management strategies by correlating the MRI features of breast cancer related to tumor morphology, heterogeneity, and angiogenesis with total RNA sequencing data. Specifically, tumor morphology was evaluated using the BI-RADS lexicon, tumor heterogeneity was assessed using tissue analysis, and the degree of gene expression according to the phenotypes of MRI variables was analyzed for different molecular subtypes of breast cancer. It was found that information about the genes differentially expressed according to the various MRI phenotypes, as well as related information about the progression or prognosis of breast cancer and for selecting breast cancer treatment methods, could be usefully employed, leading to the present disclosure.

Therefore, the present disclosure aims to provide a method for providing information about the progression or prognosis of breast cancer using MRI images of breast cancer and information for selecting methods of treatment.

### Solution to Problem

Provided according to an embodiment of the present disclosure is a method for providing information about the progression or prognosis of breast cancer using MRI images of breast cancer, the method including the following steps of:
(a) identifying the phenotype of MRI from MRI images obtained from a breast cancer patient;
(b) predicting at least one gene information differentially expressed according to the identified MRI phenotype and pathological molecular subtype; and
(c) providing information about the progression or prognosis of breast cancer from the predicted gene information.

In an embodiment of the present disclosure, the MRI phenotype is selected from a group consisting of a tumor size, a number of tumors, a tumor shape, enhancement kinetics, and a tumor texture, but with no limitations thereto.

In an embodiment of the present disclosure, the tumor size is based on the criterium that whether the diameter of the tumor is over 20 mm or 20 mm or less.

In one embodiment of the present disclosure, the number of tumors is either one or more.

In one embodiment of the present disclosure, the tumor shape includes is based on the criteria that i) whether the lesion type is mass or non-mass; ii) whether the shape of the mass-type tumor is irregular or oval to round; iii) whether the boundaries of the mass are spiculated, or circumscribed or irregular; iv) whether the internal enhancement characteristics of the mass are rim-like, or homogeneous or heterogeneous; v) whether the distribution of the non-mass type tumor is segmental, or focal, linear, regional, or diffuse; or vi) whether the internal enhancement pattern of the non-mass is clustered ring or clumped, or homogeneous or heterogeneous.

In one embodiment of the present disclosure, the enhancement kinetics are based on the criteria that i) whether the initial enhancement is fast, medium, or slow; ii) whether the delayed enhancement is plateau or washout, or persistent; or ili) whether the percentage of the washout component is over 31.31% or 31.31% or less.

In one embodiment of the present disclosure, the tumor texture is selected from a group consisting of the i) mean pixel intensity, ii) standard deviation, iii) mean of positive pixels, iv) entropy, v) kurtosis, and vi) skewness extracted from T2 images, preconstrast T1-weighed images (PreT1), and postcontrast T1-weighed images (PostT1) at the first phase of contrast injection, obtained when spatial scale filter (SSF) is 0, 2, or 5.

In one embodiment of the present disclosure, the tumor texture is PostT1-PreT1, which is the difference between selected variable values from i) to vi) of PostT1 and selected variable values from i) to vi) of PreT1, when SSF is 0, 2, or 5.

In one embodiment of the present disclosure, the gene information includes i) the type of gene; and ii) whether the gene is upregulated or downregulated.

In an embodiment of the present disclosure, if the MRI phenotype in step (a) is a mass-type lesion, it is predicted that genes such as CCL3L1, SNORA31, SNORA45, or a combination thereof will be upregulated as gene information in step (b), compared to non-mass enhancement lesion types.

In a specific embodiment of the present disclosure, if upregulation of the CCL3L1 gene is predicted as gene information in step (b), the information about the progression or prognosis of breast cancer in step (c) predicts increased migration and invasion of breast cancer cells compared to non-mass enhancing lesion types of breast cancer.

In an embodiment of the present disclosure, if the MRI phenotype in step (a) is an irregular mass lesion, the gene information in step (b) is predicted to involve downregulation of genes such as LINC01124, Y-RNA, MIR421, DEGS1, VIMP, or a combination thereof.

In a specific embodiment of the present disclosure, if downregulation of the MIR421 gene is predicted, the information about the progression or prognosis of breast cancer in step (c) predicts higher probabilities of proliferation, migration, invasion, or diagnosis as inflammatory breast cancer, and lower distant metastasis-free survival probabilities compared to round or oval mass-type breast cancer.

In an embodiment of the present disclosure, the pathological molecular subtype refers to the pathological molecular subtype of the breast cancer patient, specifically i) estrogen receptor (ER) positive or negative, ii) whether it is triple-negative breast cancer, or iii) HER2 positive or negative, but is not limited thereto.

In an embodiment of the present disclosure, if the breast cancer subtype of the patient is estrogen receptor (ER) positive and the MRI phenotype in step (a) is a mass lesion type, then the gene information in step (b) is predicted to exhibit the expression pattern as follows: genes such as SNORA31, CCL3L1, SNHG12, FTH1, MIR206, SLC39A7, CD9, or a combination thereof will be upregulated; CHD4, SOX17, SNORA30, MIR126, MIR597, or a combination thereof will be downregulated; or a combination thereof, compared to non-mass lesion type.

In a specific embodiment of the present disclosure, if such gene information is predicted, the information about the progression or prognosis of breast cancer in step (c) predicts higher possibilities of cell proliferation, resistance to chemotherapy, and potential for metastasis in ER-positive and mass-type breast cancer.

In an embodiment of the present disclosure, if the breast cancer subtype of the patient is not triple-negative breast cancer and the MRI phenotype in step (a) is a mass lesion type, then gene information in step (b) is predicted to exhibit the expression pattern such that genes such as SNORA31, CCL3L1, SNORA71B or a combination thereof will be upregulated compared to non-mass lesion types.

In a specific embodiment of the present disclosure, if such gene information is predicted, the information about the progression or prognosis of breast cancer in step (c) predicts a higher possibility of cell proliferation, migration, and metastasis for mass-type breast cancer that is not triple-negative.

In an embodiment of the present disclosure, if the breast cancer subtype of the patient is triple-negative breast cancer and the MRI phenotype in step (a) shows increased standard deviation in Pre-T1 at SSF 5, then the gene information in step (b) is predicted to exhibit expression patterns as follows: genes such as CLEC3A, SRGN, DACT1, CGA, HSPG2, ABCC5, KMT2D, FBP1, VMP1, FZD2, or a combination of these will be upregulated; PRDX4, NOP10, IGLC2, SNORA50, or a combination of these will be downregulated; or a combination thereof.

In a specific embodiment of the present disclosure, if such gene information is predicted, the information about the progression or prognosis of breast cancer in step (c) predicts higher chances of resistance to chemotherapy, metastasis, recurrence, and lower survival rates for triple-negative breast cancer with increased standard deviation in Pre-T1 at SSF 5.

In an embodiment of the present disclosure, if the breast cancer subtype of the patient is HER2 positive and the MRI phenotype in step (a) shows increased postT1_mpp at SSF 2, then the gene information in step (b) is predicted to exhibit the expression pattern such that genes such as MLKL, POTEM, or a combination thereof will be upregulated.

In a specific embodiment of the present disclosure, if such gene information is predicted, the information about the progression or prognosis of breast cancer in step (c) predicts that for HER2 positive breast cancer with increased tumor texture in postT1_mpp at SSF 2, there is a higher likelihood of increased tumor size and upregulation of the Ki-67 gene.

In an embodiment of the present disclosure, if the breast cancer subtype of the patient is HER2 positive and the MRI phenotype in step (a) shows decreased T2_mpp at SSF 5, then the gene information in step (b) is predicted to exhibit the expression pattern such that the CXCL10 gene will be upregulated.

In a specific embodiment of the present disclosure, if such gene information is predicted, the information about the progression or prognosis of breast cancer in step (c) predicts that for HER2 positive breast cancer with decreased tumor texture in T2_mpp at SSF 5, there is a higher likelihood of cell proliferation and metastasis.

According to another aspect thereof, the present disclosure provides a method for providing information for selecting a breast cancer treatment method, using breast cancer MRI images, the method including the steps of:
(a) identifying an MRI phenotype from MRI images obtained from a breast cancer patient;
(b) predicting one or more gene information differentially expressed according to the identified MRI phenotype and pathological molecular subtype; and
(c) determining a personalized breast cancer treatment method from the predicted gene information and providing information about the determined treatment method.

The method for providing information for selecting a breast cancer treatment method, using breast cancer MRI images in accordance with the present disclosure shares the configurations such as identifying the MRI phenotype, predicting the gene information, etc. with the method for providing information about progression or prognosis of breast cancer, using breast cancer MRI images described above according to an embodiment of the present disclosure, except for step (c) of determining a personalized breast cancer treatment method from the predicted gene information and providing information about the determined treatment method. Therefore, the overlapping content therebetween is mutually exchangeable, and the description of overlapping content is omitted to avoid complexity herein.

According to another aspect thereof, the present disclosure provides a method for treating breast cancer, the method including the steps of:
(a) identifying an MRI phenotype from MRI images obtained from a breast cancer patient;
(b) predicting one or more gene information differentially expressed according to the identified MRI phenotype and pathological molecular subtype;
(c) determining a personalized breast cancer treatment method from the predicted gene information; and
(d) treating the breast cancer patient with the treatment method determined in step (c).

In step (d), the breast cancer patient can be treated with anti-estrogen therapy on the basis of the determination of step (c).

In a specific embodiment of the present disclosure, the anti-estrogen therapy includes administering a selective estrogen receptor modulator (SERM) or an aromatase inhibitor.

Examples of the selective estrogen receptor modulators include tamoxifen and toremifene, but are not limited thereto.

Examples of aromatase inhibitors include exemestane, anastrozole, and letrozole, but are not limited thereto.

In step (d), the breast cancer patient can be treated with adjuvant chemotherapy, prophylactic mastectomy, or a combination thereof on the basis of the determination of step (c),

The adjuvant chemotherapy includes administering i) cyclophosphamide, methotrexate, and 5-fluorouracil; ii) cyclophosphamide, doxorubicin, and 5-fluorouracil; iii) cyclophosphamide and doxorubicin; iv) cyclophosphamide, doxorubicin, and paclitaxel; v) docetaxel, doxorubicin, and cyclophosphamide, but with no limitations thereto, and the dosage, schedule, intervals, and cycles of these chemotherapeutic drugs are well known to clinicians in the field and are not limited to the mentioned drugs and can be appropriately selected and used under the judgment of the clinician.

The treatment method for breast cancer of the present disclosure shares the configurations such as identifying the MRI phenotype, predicting the gene information, etc. with the method for method for providing information for selecting a breast cancer treatment method, using breast cancer MRI images, described above according to an embodiment of the present disclosure, except for step (d) of treating the breast cancer patient. Therefore, the overlapping content therebetween is mutually exchangeable, and the description of overlapping content is omitted to avoid complexity herein.

According to another aspect thereof, the present disclosure provides a breast cancer treatment method selection system comprising the following components:
(a) a database where to search and extract information about genes related to breast cancer treatment;
(b) a communication unit capable of accessing the database;
(c) a first decision module for determining a tumor phenotype, using MRI images obtained from the patient;
(d) a second decision module for determining one or more gene information related to breast cancer by using the tumor phenotype;
(e) a third decision module for determining a treatment method for breast cancer from the derived gene information; and
(f) a display for showing the decision values determined by at least one of the decision modules.

In one embodiment of the present disclosure, the database may be one that has been conventionally constructed, constructed by the inventors, or a combination thereof.

In one embodiment of the present disclosure, the tumor phenotype determined in the first decision module is as described in the other aspects of the present disclosure.

In an embodiment of the present disclosure, the one or more gene information related to breast cancer determined in the second decision module is as described in the other aspects of the present disclosure.

In an embodiment of the present disclosure, the treatment method for breast cancer determined in the third decision module is as described in the other aspects of the present disclosure.

The system according to the present disclosure may further include a user interface that accesses a database where to search and extract information about treatment methods applicable to breast cancer patients and genes related to the treatment methods, extracts related information accordingly, and provides the user with information about the personalized treatment method.

In the system according to the present disclosure, the server containing the database or the access information thereof, the produced information, and the user interface device or terminal connected thereto may be used in conjunction with each other.

In the system according to the present disclosure, the user interface device or terminal may request information about personalized breast cancer treatment methods based on gene expression changes according to the phenotype of breast cancer from the server, receive the result, and/or store the request and result. The user interface device or terminal may be a terminal, such as a smart phone, a PC (Personal Computer), a tablet PC, a personal digital assistant (PDA), and a web pad, which includes a memory means and has a mobile communication function with a calculation ability using a microprocessor.

In the system according to the present disclosure, the server is a means for providing an access to the database and is connected to the user interface or terminal through the communication unit so as to exchange various kinds of information.

Herein, the communication unit may include not only communication in the same hardware but also a local area network (LAN), a metropolitan area network (MAN), a wide area network (WAN), the Internet, 2G, 3G and 4G mobile communication networks, Wi-Fi, Wibro, and the like, and may use any communication method regardless of whether it is wired or wireless.

The database may be directly installed in the server and may also be connected to various life science databases accessible via the Internet depending on a purpose.

The method according to the present disclosure may be implemented by hardware, firmware, software, or combinations thereof.

If the method is implemented by software, a storage medium may include any storage or transmission medium readable by a device such as a computer. For example, the computer-readable medium may include a ROM (read only memory); a RAM (random access memory); a magnetic disc storage medium; an optical storage medium; a flash memory device; and other electric, optical or acoustic signal transmission medium.

### Advantageous Effects of Invention

The present disclosure provides a method for providing information about the progression or prognosis of breast cancer, as well as information for selecting breast cancer treatment methods, using breast cancer MRI images. When using the method of the present disclosure, gene information can be predicted using non-invasive MRI imaging, and accordingly, it can be usefully used to provide information about the progression or prognosis of breast cancer and information for selecting breast cancer treatment methods.

### BRIEF DESCRIPTION OF DRAWINGS

FIG. 1 is a flowchart of recruiting study participants. MRI=magnetic resonance imaging.

FIGS. 2a, 2b and 2c are MRI images of invasive ductal carcinoma in a 61-year-old-woman. (2a) Tumor morphology assessments were performed on T2-weighed MRI, and pre-and postcontrast T1-weighed MRI using the BI-RADS lexicon. An irregularly shaped, marginated, and heterogeneous enhancing mass (arrows) are seen. (2b) Texture analysis was performed within a region of interest using SSFs of 0 (unfiltered texture), 2 (fine-filtered texture), and 5 (coarse-filtered texture). (2c) CAD-color overlay map exhibits tumor enhancement kinetics. Red, green, and blue regions stand for washout, plateau, and persistent enhancement pattern, respectively. BI-RADS=Breast Imaging-Reporting and Data System, SSF=spatial scale filter, CAD=computer-aided diagnosis.

FIG. 3a shows genes differentially expressed according to lesion types of MRI phenotypes in ER-positive breast cancer patients as analyzed by a heat map.

FIG. 3b shows genes differentially expressed according to MRI texture SSF 5 PreT1 standard deviation in triple-negative breast cancer as analyzed by a heat map.

FIG. 4 is a diagram of gene network analysis obtained from Ingenuity Pathway Analysis with an input of top 100 genes (Q value <. 1) differentially expressed according to MRI phenotype (P<. 05).

FIGS. 5a and 5b show canonical pathways of breast cancer-related genes differentially expressed according to lesion types in ER-positive breast cancers.

### BEST MODE FOR CARRYING OUT THE INVENTION

A better understanding of the present disclosure may be obtained through the following examples which are set forth to illustrate, but are not to be construed to limit, the present disclosure.

### Examples

Throughout the description, the term “%” used to express the concentration of a specific material, unless otherwise particularly stated, refers to (wt/wt) % for solid/solid, (wt/vol) % for solid/liquid, and (vol/vol) % for liquid/liquid.

### Experimental Materials and Methods

Research Participants

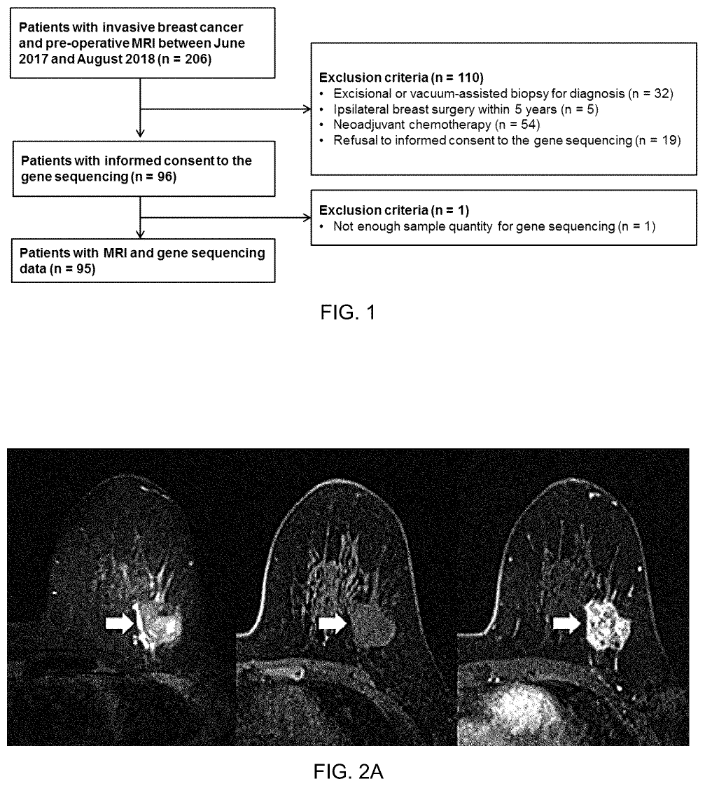

This prospective study was approved by the Institutional Review Board, and all participants provided written informed consent before participating. From June 2017 to August 2018, 206 consecutive participants with pathologically proven invasive breast cancer underwent breast MRI. Of the 206 participants, 111 were excluded for at least one of the following reasons: (a) excisional or vacuum-assisted biopsy for diagnosis (n=32); (b) ipsilateral breast surgery within 5 years (n=5); (c) prior chemotherapy (n=54); (d) refusal of informed consent to the gene sequencing (n=19); and (e) insufficient sample quantity for RNA testing (n=1). Ultimately, 95 participants with invasive breast cancers were included in this study (FIG. 1). Next-generation sequencing was performed using the whole genomic RNA obtained from surgical specimens.

FIG. 1 is a flowchart concerning the recruitment of participants for this study. MRI=magnetic resonance imaging.

### Characteristics of Study Participants

All the 95 patients (all female, average age 53+11 [standard deviation]) with 95 breast cancer (average size, 24.9 mm+13.0) were included in this study. The lesion characteristics of the 95 breast cancers are summarized in Table 1.

TABLE 1
Characteristics of Research Participants

| Characteristics | Values | |
|---|---|---|
| Age (years) | 53 ± 10 | (25-81) |
| Lesion size (mm) | 24.9± | (6-72) |

| | |
|---|---|
| Histologic type | |
| Invasive ductal carcinoma | 81 |
| Mucinous carcinoma | 4 |
| Invasive lobular carcinoma | 3 |
| Invasive micropapillary carcinoma | 3 |
| Tubular carcinoma | 2 |
| Medullary carcinoma | 1 |
| Metaplastic carcinoma | 1 |
| Molecular subtype | |
| Luminal A | 42 |
| Luminal B | 25 |
| HER2-enriched | 13 |
| Triple negative | 15 |

Note
Unless otherwise specified, the data are the number of cancers. Average data are ± standard deviation. Data in parentheses are ranges. All participants (n = 95) were female. HER2 = human epidermal growth factor receptor 2.

A 3 T MRI system (MAGNETOM Skyra; Siemens Healthineers, Erlangen, Germany) with a dedicated 4-channel breast coil was used. Bilateral transverse fat-suppressed T2-weighed images (repetition time msec/echo time msec, 4050/56; matrix, 307 X 384; field of view, 340×340 mm; flip angle 120°; reconstruction voxel size, 0.44 X 0.44×3 mm; slice thickness, 3 mm) and transverse fat-suppressed T1-weighed images (3.44/1.36; matrix, 320 X 320; field of view, 320×320 mm; reconstruction voxel size, 1 X 1×1 mm; slice thickness, 1 mm) were acquired. The T1-weighed images included one precontrast image (photographed at two different flip angles (2° and 9º) in the transverse plane encompassing the entire tumor volume) and five postcontrast images (I.V. injection of gadoterate meglumine at a dose of 0.2 mL/kg, followed by a 30-mL saline flush, photographed at 93, 180, 268, 356, and 443 s after the start of contrast agent injection).

Image evaluation was performed by two radiologists. Tumor morphology was evaluated according to the BI-RADS lexicon, and tumor heterogeneity and angiogenetic characteristics were evaluated with commercially available software. For texture analysis, a filtration histogram technique (TexRAD; Feedback Medical Ltd., Cambridge, UK) was employed. The enhancement kinetics was assessed using a CAD

MRI Analysis system (CADstream, version 6.0; Confirma, Kirkland, Wash).

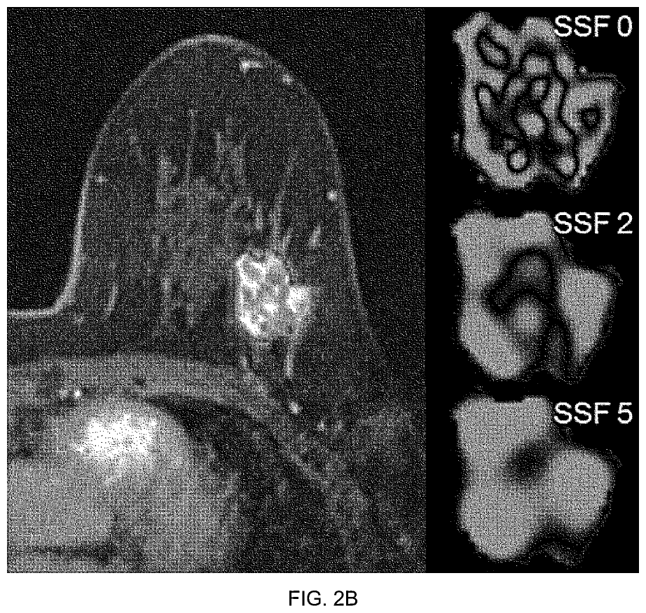

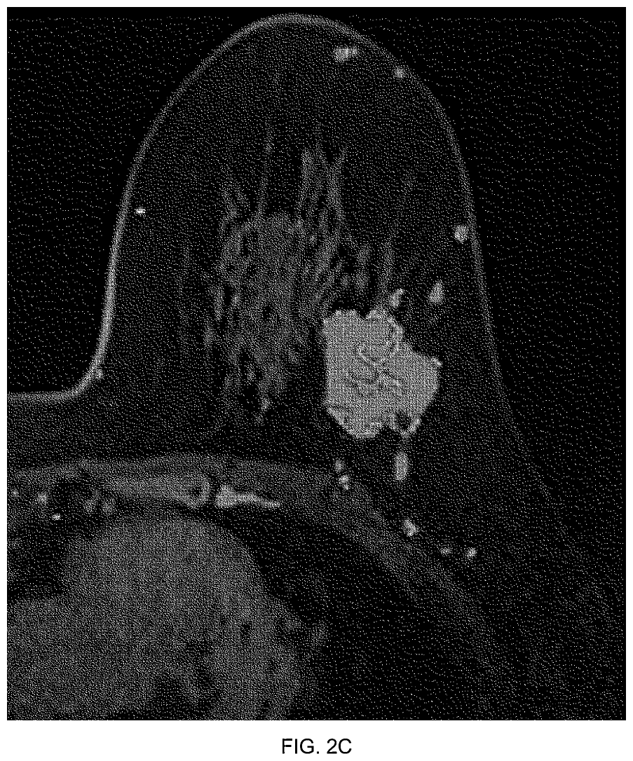

FIGS. 2a to 2c are MRI images of invasive ductal carcinoma in a 61-year-old-woman. (2a) Tumor morphology assessments were performed on T2-weighed MRI, and pre-and postcontrast T1-weighed MRI using the BI-RADS lexicon. An irregularly shaped, marginated, and heterogeneous enhancing mass (arrows) appear. (2b) Texture analysis was performed within a region of interest using SSFs of 0 (unfiltered texture), 2 (fine-filtered texture), and 5 (coarse-filtered texture). (2c) CAD-color overlay map exhibits tumor enhancement kinetics. Red, green, and blue regions stand for washout, plateau, and persistent enhancement pattern, respectively. BI-RADS=Breast Imaging-Reporting and Data System, SSF=spatial scale filter, CAD=computer-aided diagnosis.

Depending on tumor morphology, the lesion type was divided into mass or non-mass enhancement. In masses, shape, margin, and internal enhancement characteristics were evaluated and in non-mass enhancement, distribution, and internal enhancement patterns were evaluated. For texture analysis, T2, precontrast T1-weighed images (PreT1), and postcontrast T1-weighed images (PostT1) at the first phase of contrast injection were used. The following six texture parameters were extracted according to various spatial scale filters (SSFs) ranging in pixel scale from unfiltered texture (SSF 0) to 2 (2 mm pixel scale) and 5 (5 mm pixel scale): i) mean pixel intensity, ii) standard deviation, iii) mean of positive pixels, iv) entropy, v) kurtosis, and vi) skewness.

Texture functions for PostT1-PreT1 were obtained by subtracting each variable value of PreT1 from each parameter value of PostT1. A total of 72 quantified texture parameters were acquired. In the CAD analysis, the initial [slow (pixel value increase <50%), medium (pixel value increase 50-100%), or fast (pixel value increase >100%)] and delayed phase enhancement patterns [washout; at least 10% decrease), plateau (within 10% increase or decrease), or persistent (at least 10% increase)] and the proportion of washout components for each tumor were extracted.

Differentially Expressed Genes Associated with Breast Cancer According to MRI Phenotypes

Eighteen genes were expressed differentially according to the three MRI phenotypes with the standard of P<. 05, Q<. 01, and log 2FC >2.0 or <−2.0.

Of them, three genes were upregulated and 15 were downregulated. Among 18 differentially expressed genes, three were protein-coding genes, five were noncoding genes, and ten were pseudogenes or unidentified genes. The five non-coding RNA gene include two small nucleolar RNAs, one microRNA, one long non-coding RNA, and one Y-RNA.

In Table 22, eight of the differentially expressed protein-coding and noncoding genes according to MRI phenotypes, except for the pseudogenes and unidentified genes, are summarized.

TABLE
Differentially expressed genes associated with
breast cancer according to MRI phenotypes

| MRI phenotype | Genes | Log2FC | P value | Q value |
|---|---|---|---|---|
| Lesion type | CCL3L1 | 2.81 | .001 | .063 |
| | SNORA31 | 2.77 | <.001 | .053 |
| | SNORA45 | 2.81 | <.001 | .047 |
| Mass shape | LINC01124 | −2.09 | <.001 | .001 |
| | Y-RNA | −2.13 | <.001 | .005 |
| | MIR421 | −2.57 | <.001 | .005 |
| | DEGS1 | −2.66 | <.001 | .003 |
| | VIMP | −2.76 | .001 | .096 |

As seen in Table 2, breast cancer with mass lesion type showed the upregulation of CCL3L1 (log 2fc=2.81, P=. 001, Q=. 063), which is involved in the migration and invasion of breast cancer cells, compared with non-mass enhancement type.

In mass lesions, irregular shape showed the downregulation of MIR421 (log 2FC=−2.57, P<. 001, Q=. 005), which is involved in cell proliferation, migration, invasion, and inflammatory breast cancer, poor metastasis-free survival in breast cancer, compared with circular/oval shapes.

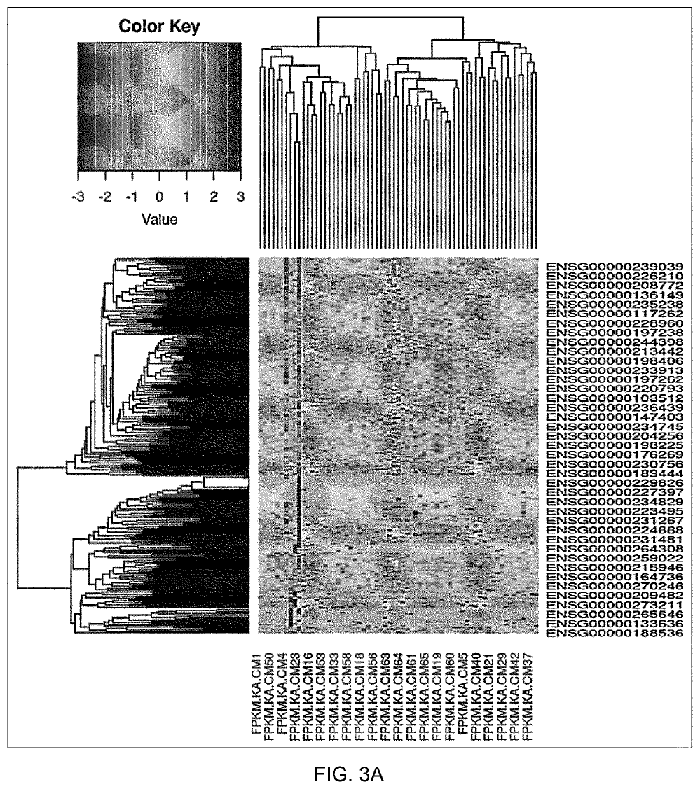

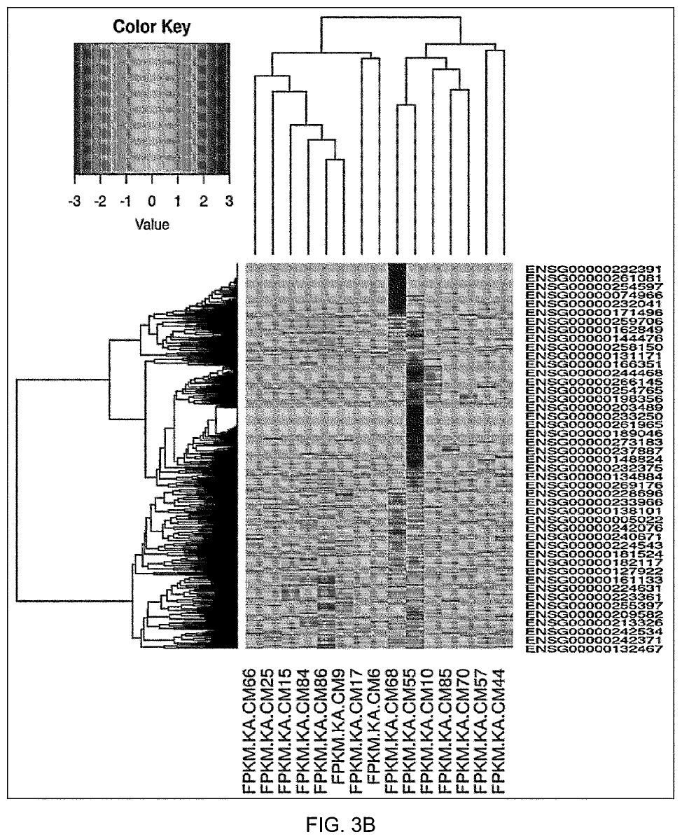

Additional analysis was made of gene expression according to MRI phenotype for each molecular subtype of breast cancer based on the expression of estrogen receptor (ER) or human epidermal growth factor receptor 2 (HER2) genes. The results are presented in Table 3 and FIGS. 3a-3d.

Table 3 summarizes key genes associated with breast cancer that are differentially expressed according to MRI phenotype for each subtype of breast cancer.

TABLE 3
Key genes associated with breast cancer differentially expressed
according to MRI phenotype for each subtype of breast cancer

| Breast cancer | | | | P | Q |
|---|---|---|---|---|---|
| subtype | MRI phenotype | Genes | Log2FC | value | value |
| ER-positive | Lesion type | SNORA31 | 5.93 | <.001 | .003 |
| | | CCL3L1 | 4.40 | .001 | .047 |
| | | SNHG12 | 3.43 | .002 | .066 |
| | | FTH1 | 3.03 | <.001 | <.001 |
| | | MIR206 | 2.86 | .002 | .053 |
| | | SLC39A7 | 2.65 | .002 | .048 |
| | | CD9 | 2.04 | .003 | .080 |
| | | CHD4 | −2.21 | <.001 | .015 |
| | | SOX17 | −2.28 | <.001 | .003 |
| | | SNORA30 | −2.44 | .001 | .044 |
| | | MIR126 | −3.63 | .001 | <.001 |
| | | MIR597 | −8.05 | <.001 | <.001 |
| ER-positive or | Lesion type | SNORA31 | 3.34 | <.001 | .04 |
| HER2-positive | | CCL3L1 | 3.08 | .001 | .05 |
| | | SNORA71B | 2.92 | .001 | .06 |
| HER2-positive | SSF 2 postT1_{—} | MLKL | 2.20 | <.001 | .064 |
| | mpp | | | | |
| HER2-positive | SSF 5 T2_mpp | CXCL10 | 3.27 | <.001 | .08 |
| Triple-negative | SSF 5 preT1_SD | CLEC3A | 4.50 | <.001 | .036 |
| | | SRGN | 3.72 | <.001 | .062 |
| | | DACT1 | 3.61 | <.001 | .002 |
| | | CGA | 2.89 | <.001 | .035 |
| | | HSPG2 | 2.85 | .002 | .084 |
| | | ABCC5 | 2.36 | <.001 | .007 |
| | | KMT2D | 2.35 | .001 | .035 |
| | | FBP1 | 2.29 | <.001 | .035 |
| | | VMP1 | 2.26 | <.001 | .037 |
| | | FZD2 | 2.06 | .002 | .085 |
| | | PRDX4 | −2.80 | .002 | .094 |
| | | NOP10 | −3.45 | .001 | .052 |
| | | IGLC2 | −6.18 | <.001 | .016 |
| | | SNORA50 | −9.25 | .001 | .063 |

As seen in Table 3 and FIGS. 3a-3b, in ER-positive cancers, a total of 31 genes (SNORA31, CCL3L1, SNHG12, FTH1, MIR206, SLC39A7, CD9, etc.) and a total of 22 genes (CHD4, SOX17, SNORA30, MIR126, MIR597, etc.) were upregulated and downregulated, respectively, for mass-type cancers on MRI images. Expression of these genes is predicted to increase the likelihood of cell proliferation, chemoresistance, and metastasis in ER-positive, tumor-type breast cancer.

Additionally, in breast cancer that is not triple-negative (ER positive or HER2 positive) and has a mass-type MRI appearance, three genes (SNORA31, CCL3L1, SNORA71B) were upregulated, and five genes were downregulated. The expression of these genes suggests that there is a higher likelihood of cell proliferation, migration, invasion, and metastasis in breast cancer that is not triple-negative and is mass-type.

As shown in Table 3 and FIGS. 3c-3d, in triple-negative breast cancer with increased standard deviation in preT1 at SSF 5 as the MRI phenotype, 29 genes (including CLEC3A, SRGN, HSPG2, ABCC5, KMT2D, FBP1, VMP1, FZD2) were upregulated, and 14 genes (including SNORA5, IGLC2, PRDX4) were downregulated. The expression of these genes predicts a higher likelihood of chemoresistance, metastasis, recurrence, and poor survival in triple-negative breast cancer with increased standard deviation in preT1 at SSF 5.

In HER2 positive tumors with increased postT1_mpp at SSF 2, two genes (MLKL, POTEM) were upregulated. The expression of these genes suggests a higher likelihood of increased tumor size and increased Ki-67, reflecting the degree of tumor cell proliferation in HER2 positive breast cancer with increased postT1_mpp at SSF 2.

In HER2 positive tumors with decreased T2_mpp at SSF 5, one gene (CXCL10) was upregulated. The expression of this gene suggests a higher likelihood of cell proliferation and metastasis in HER2 positive breast cancer with decreased T2_mpp at SSF 5.

RNA Sequencing and Analysis

After calculating the total RNA concentration using Quant-IT RiboGreen (Invitrogen, Carlsbad, CA), sequencing libraries were constructed for 100 ng of total RNA. Paired-end (2×100 base pairs) sequencing was performed using the Illumina NovaSeq6000 sequencing system. In addition, low-quality and adapter sequences were trimmed from paired-end reads using Trim Galore software (version 0.6.5) and Cutadapt (version 1.15). To extract RNA sequence variants, the Genome Analysis Toolkit, which is the best practice workflow for single nucleotide polymorphism and InDel calling, was used. Briefly, the Spliced Transcripts Alignment to a Reference 2-pass method was used to align the trimmed reads to the human reference genome (hg19). The resulting Spliced Transcripts Alignment files were then processed using Picard tools to add read group information, sort, mark duplicates, and index. As a final step, annotation of RNA variants was done using Annotate Variation. Enhanced function annotation and pathway analysis were subsequently performed using Ingenuity Pathway Analysis software (Ingenuity Systems, Redwood City, CA).

Gene Network Identification

The gene network analysis was performed by the Ingenuity Pathway Analysis using top 100 differentially expressed genes (Q<. 1) according to MRI phenotypes (P<. 05).

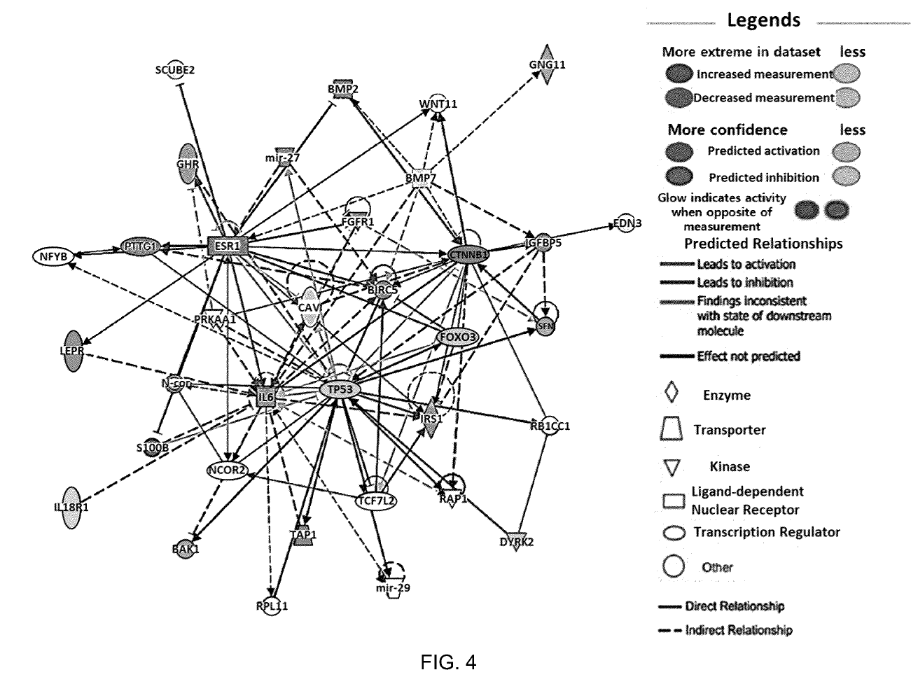

The results are depicted in FIG. 4.

As shown in FIG. 4, in one of the top networks for the lesion type in ER-positive cancers, the genes ESR1, BIRC5, CAV1, FGFR1, IL6, MIR27, and PTTG1 were upregulated with direct or indirect interactions between them. The genes have been found to be associated with increased anti-estrogen resistance, metastasis, and poor survival in ER-positive breast cancer. The functions of the network included cell cycle, cellular growth, and proliferation, with a score of 11.

Enriched Functional Annotation

Enriched functional annotation was obtained from Ingenuity Pathway Analysis with an input of top 100 differentially expressed genes (Q value <. 1) according to MRI phenotypes (P<. 05).

The results are given in Tables 4 and 5.

TABLE 4
Enriched function of genes differentially expressed
according to lesion types in ER-positive tumors

| Diseases or functions annotation | P value | Genes |
|---|---|---|
| Signal transduction | <.001 | ADRA1D, CRHBP, EDN3, GNG11, IGF1, |
| | | IL18R1, IL1RL1, RCVRN |
| Cell division of breast cancer cell | .001 | IGF1 |
| lines | | |
| Migration of breast cancer cell | .002 | DPP4, IGF1, SLC16A4, WNT11 |
| lines | | |
| Apoptosis of mammary cells | .002 | IGF1 |
| Proliferation of stromal cell lines | .002 | IGF1 |
| Transition of breast cancer cell | .003 | IGF1 |
| lines | | |
| Arrest in G0/G1 phase transition | .008 | IGF1 |
| of breast cancer cell lines | | |
| Development of adenocarcinoma | .009 | ACACB, ADRA1D, CRHBP, DPP4, DYRK2, |
| | | FER1L5, IGF1, IL18R1, LARP7, MAP6, |
| | | RCVRN, SCN3A, SCN7A, SCUBE2, |
| | | SLC16A4, WNT11, ZNF136 |
| Breast or gastric cancer | .01 | ACACB, ADRA1D, CRHBP, DPP4, DYRK2, |
| | | FER1L5, HMX1, IFNA10, IGF1, IL1RL1, |
| | | LARP7, MAP6, SCN3A, SCN7A, SCUBE2, |
| | | SLC16A4, WNT11 |
| Breast or gynecological cancer | .011 | ACACB, ADRA1D, CRHBP, DPP4, DYRK2, |
| | | FER1L5, IGF1, IL18R1, IL1RL1, LARP7, |
| | | MAP6, RCVRN, SCN3A, SCN7A, SCUBE2, |
| | | SLC16A4, WNT11, ZNF136 |
| Breast or ovarian carcinoma | .011 | ACACB, ADRA1D, CRHBP, DPP4, FER1L5, |
| | | IGF1, IL1RL1, MAP6, SCN3A, SCN7A, |
| | | SCUBE2, WNT11 |
| Anoikis of breast cell lines | .012 | IGF1 |
| Mitogenesis of breast cancer cell | .015 | IGF1 |
| lines | | |
| Neoplasia of tumor cell lines | .015 | DPP4, IL1RL1 |
| Chemotaxis | .016 | DPP4, EDN3, IGF1 |
| Proliferation of endocrine cell | .019 | IGF1 |
| lines | | |
| G-protein signaling, coupled to | .019 | ADRA1D |
| cAMP nucleotide second | | |
| messenger | | |
| Transactivation of RNA | .02 | ACACB, DPP4, IGF1 |
| Chemotaxis of breast cancer cell | .02 | IGF1 |
| lines | | |
| Expression of rRNA | .023 | IGF1 |
| Anoikis of breast cancer cell lines | .025 | IGF1 |
| Proliferation of stromal cells | .026 | IGF1 |

In Table 4, the functions associated with breast cancer or general cancer were annotated primarily concerning the MRI lesion type in ER-positive cancers

TABLE 5
Enhanced function of differentially expressed genes according to
preT1_standard deviation at SSF 5 in triple-negative breast cancer

| | P | |
|---|---|---|
| Diseases or functions annotation | value | Genes |
| Progesterone receptor signaling pathway | .003 | TRERF1, YAP1 |
| Cell cycle progression of breast cancer cell | .007 | CYP3A4, ESR1, |
| lines | | TNC, YAP1 |
| Clumping by breast cancer cell lines | .011 | ESR1 |
| Exit from cell cycle progression of epithelial | .011 | YAP1 |
| cell lines | | |
| Arrest in mitosis of tumor cells | .011 | ACRBP |
| Adhesion of stromal cell lines | .011 | SRGN |
| Progressive unresectable estrogen receptor | .011 | ESR1 |
| positive HER2 negative breast cancer | | |
| Unresectable estrogen receptor positive HER2 | .011 | ESR1 |
| negative breast adenocarcinoma | | |
| Binding of basic transcription element | .011 | KLF13 |
| Angiogenesis of malignant tumor | .011 | CCN4 |
| Apocrine breast carcinoma | .011 | ESR1 |
| Metastatic estrogen receptor mutation positive | .011 | ESR1 |
| HER2 negative breast adenocarcinoma | | |
| G-protein signaling, coupled to cAMP | .015 | ADRA1B, |
| nucleotide second messenger | | ADRA1D |
| Migration of adenocarcinoma cell lines | .06 | SATB2, SRGN, |
| | | YAP1 |
| Growth of mammary tumor | .02 | CCN4, ESR1 |

In Table 5 above, preT1_standard deviation in the tumor texture SSF 5 in triple-negative breast cancer is annotated as a function related to breast cancer or general cancer.

### Canonical Pathway

Canonical pathway analysis was performed using the Ingenuity Pathway Analysis with an input of top 100 differentially expressed genes (Q value <. 1) according to MRI phenotypes (P<. 05).

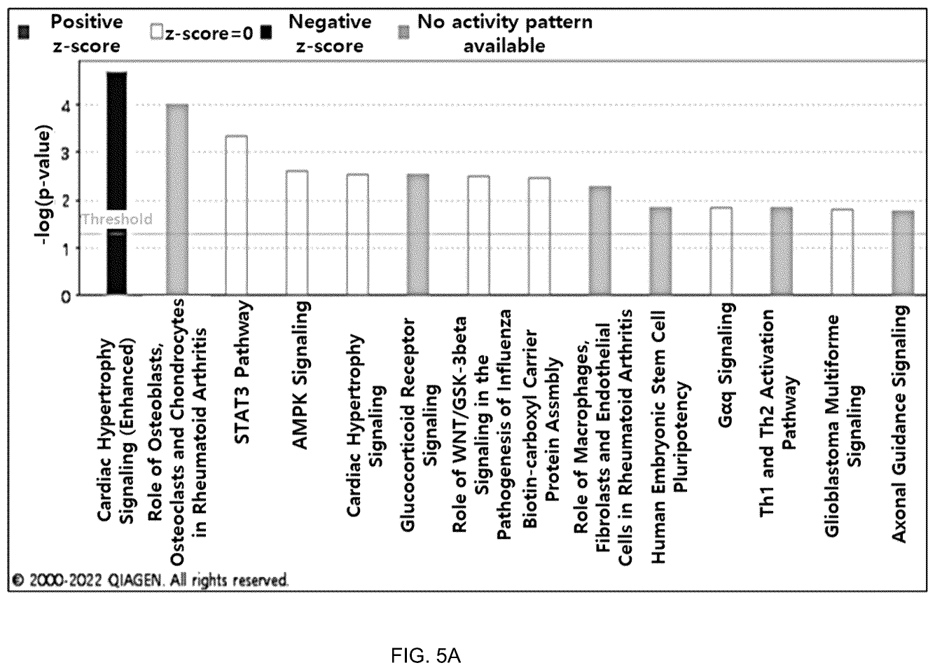

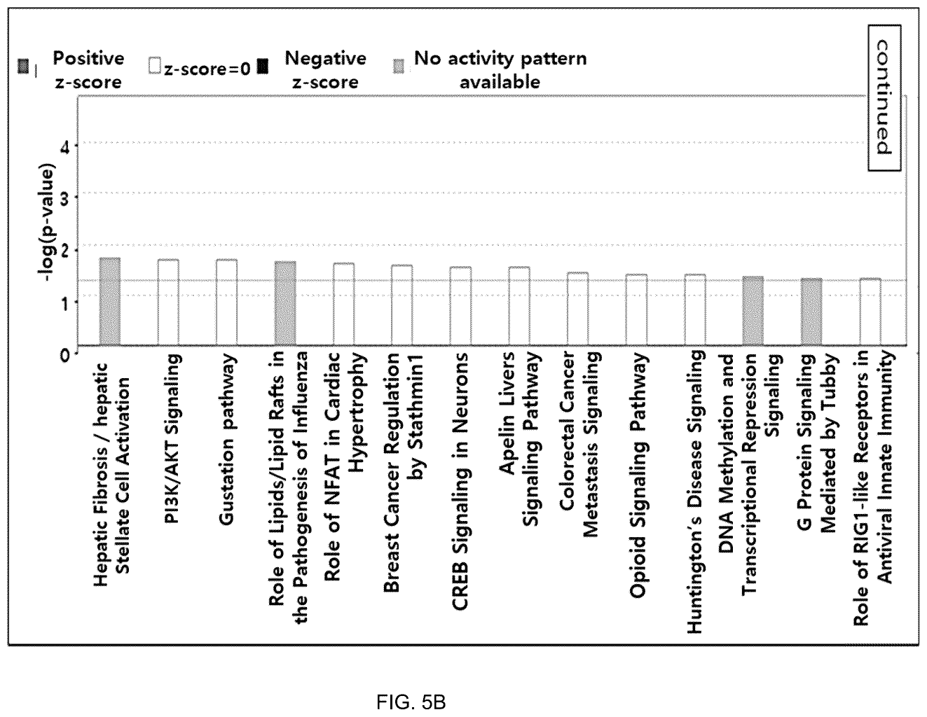

The results are depicted in Table 6 and FIG. 5.

TABLE 6
Canonical pathway of breast cancer-genes differentially
expressed according to lesion types

| | −log(p- | | |
|---|---|---|---|
| classical pathway | value) | Ratio | Genes |
| STAT3 Pathway | 3.34 | 0.02 | IGF1, IL18R1, IL1RL1 |
| AMPK Signaling | 2.61 | 0.01 | ACACB, ADRA1D, GNG11 |
| Glucocorticoid Receptor | 2.54 | <.01 | IFNA10, IGF1, IL18R1, |
| Signaling | | | IL1RL1 |
| Th1 and Th2 Activation | 1.82 | 0.01 | IL18R1, IL1RL1 |
| Pathway | | | |
| PI3K/AKT Signaling | 1.69 | 0.01 | IL18R1, IL1RL1 |
| Breast Cancer Regulation | 1.57 | <.01 | ADRA1D, GNG11, IGF1 |
| by Stathmin1 | | | |

As shown in Table 6 and FIG. 5, in ER-positive tumors, the lesion types on MRI were associated with the classical pathway of breast cancer-related genes.

### Statistical Analysis

Differential expression of individual genes between the two groups of each MRI phenotype was analyzed using Tablemaker (version 2.1.1) and Ballgown R package (version 2.22.0). Fragments per kilobase of transcript per million mapped reads were used to estimate the gene expression level. The P value for differential expression was extracted using a parametric F test comparing nested linear models. The Ballgown stattest function was used to calculate the log twofold change (log 2FC) of the gene expression between two groups of each MRI phenotype. Finally, differential gene expression results were visualized using a volcano plot and heat map in R.

### Review

1. In this prospective study, the present inventors have correlated the multiparametric MRI phenotypes of breast cancer with whole RNA sequencing data. An examination was made of genes that are differentially expressed in breast cancer based on the shape and texture analysis using BI-RADS lexicon in MRI, focusing on breast cancer heterogeneity and angiogenesis.

2. For overall breast cancer, differential expression was observed in 7 genes based on the lesion type and in 11 genes based on the shape of the breast cancer mass. The CCL3L1 gene, which increases migration and invasion of breast cancer cells, was upregulated in cases where the breast cancer appeared as a mass compared to non-mass-enhancing lesions. In breast masses, the MIR421 gene, related to cell proliferation, migration, invasion, and associated with inflammatory breast cancer and poor metastasis-free survival, was downregulated in irregularly shaped masses compared to round/oval masses. This suggests that breast cancer appearing in round/oval mass forms might be more aggressive and have a poorer prognosis.

3. In ER-positive breast cancer, when the lesion type is a mass, genes related to proliferation, migration, and invasion of breast cancer cells such as SNHG12, MIR206 were upregulated, and SLC39A7 related to worse overall survival and CD9 associated with chemoresistance were also upregulated. Conversely, tumor suppressor genes known for lower metastasis potential and shorter overall survival, MIR597, MIR126, were downregulated. This suggests that mass-type ER-positive breast cancers might be more aggressive, with increased drug resistance and metastasis potential, leading to lower survival rates.

4. In network analysis of ER-positive tumors based on MRI lesion type, genes such as ESR1, BIRC5, CAV1, FGFR1, IL6, MIR27, PTTG1 showed direct and indirect interactions with each other while being upregulated. These genes are associated with increased anti-estrogen resistance, metastasis, and poor survival. The network score was 11, including cell cycle, cellular growth, and proliferation. This result also indicates that mass-type ER-positive tumors could be related to drug resistance, metastasis, and lower survival rates.

5. In cases of triple-negative breast cancer where the tumor texture in MRI phenotype shows increased standard deviation in preT1 at SSF 5, genes associated with metastasis, chemoresistance, recurrence, and poor survival, such as CLEC3A, SRGN, HSPG2, ABCC5, KMT2D, FBP1, VMP1, and FZD2, were upregulated. Conversely, genes associated with higher survival rates, such as PRDX4 and IGLC2, were downregulated. This suggests that in triple-negative breast cancer with increased internal heterogeneity (as indicated by increased standard deviation in preT1 at SSF 5), there may be an increased likelihood of metastasis, chemoresistance, recurrence, and lower survival rates.

6. In HER2 positive tumors where the tumor texture at SSF 2 in postT1_mpp is increased, indicating greater angiogenesis and contrast enhancement, the MLKL gene was upregulated. This suggests a possibility of the breast cancer having a larger tumor size and increased cell proliferation, as indicated by increased Ki-67.

7. In HER2 positive tumors where the tumor texture at SSF 5 in T2_mpp is decreased, indicating increased cell density in breast cancer, the CXCL10 gene was upregulated. This suggests an increased likelihood of cell proliferation and metastasis in breast cancer.

8. Based on the results from 3. to 6., the expression of specific MRI phenotypes in each breast cancer subtype may be related to the differential expression of various types of genes, and these genes might potentially be used as targets for therapy in the future. This implies that MRI imaging could contribute to the evolution of breast cancer treatment towards more targeted, personalized therapies.

What is claimed is:

1. A method for providing information about progression or prognosis of breast cancer using MRI images of breast cancer, the method comprising the steps of:
(a) identifying the phenotype of MRI from MRI images obtained from a breast cancer patient;
(b) predicting at least one gene information differentially expressed according to the identified MRI phenotype and pathological molecular subtype; and
(c) providing information about the progression or prognosis of breast cancer from the predicted gene information.

2. The method of claim 1, wherein the MRI phenotype is selected from a group consisting of a tumor size, a number of tumors, a tumor shape, enhancement kinetics, and a tumor texture.

3. The method of claim 2, wherein the tumor size is based on the criterium that whether the diameter of the tumor is over 20 mm or 20 mm or less.

4. The method of claim 2, wherein the number of tumors is either one or more.

5. The method of claim 2, wherein the tumor shape includes is based on the criteria that
i) whether the lesion type is mass or non-mass;
ii) whether the shape of the mass-type tumor is irregular or oval to round;
iii) whether the boundaries of the mass are spiculated, or circumscribed or irregular;
iv) whether the internal enhancement characteristics of the mass are rim-like, or homogeneous or heterogeneous;
v) whether the distribution of the non-mass type tumor is segmental, or focal, linear, regional, or diffuse; or
vi) whether the internal enhancement pattern of the non-mass is clustered ring or clumped, or homogeneous or heterogeneous.

6. The method of claim 2, wherein the enhancement kinetics are based on the criteria that i) whether the initial enhancement is fast, medium, or slow; ii) whether the delayed enhancement is plateau or washout, or persistent; or iii) whether the percentage of the washout component is over 31.31% or 31.31% or less.

7. The method of claim 2, wherein the tumor texture is selected from a group consisting of i) mean pixel intensity, ii) standard deviation, iii) mean of positive pixels, iv) entropy, v) kurtosis, and vi) skewness extracted from T2 images, preconstrast T1-weighed images (PreT1), and postcontrast T1-weighed images (PostT1) at a first phase of contrast injection, obtained when spatial scale filter (SSF) is 0, 2, or 5.

8. The method of claim 2, wherein the tumor texture is PostT1-PreT1, which is the difference between selected variable values from i) to vi) of PostT1 and selected variable values from i) to vi) of PreT1, when SSF is 0, 2, or 5.

9. The method of claim 1, wherein the gene information comprises i) the type of gene; and ii) whether the gene is upregulated or downregulated.

10. The method of claim 1, wherein if the MRI phenotype in step (a) is a mass-type lesion, it is predicted that genes such as CCL3L1, SNORA31, SNORA45, or a combination thereof will be upregulated as gene information in step (b), compared to non-mass enhancement lesion types.

11. The method of claim 1, wherein if the MRI phenotype in step (a) is an irregular mass lesion, the gene information in step (b) is predicted to involve downregulation of LINC01124, Y-RNA, MIR421, DEGS1, VIMP, or a combination thereof.

12. The method of claim 1, wherein if the breast cancer subtype of the patient is estrogen receptor (ER) positive and the MRI phenotype in step (a) is a mass lesion type, then the gene information in step (b) is predicted to exhibit the expression pattern of: upregulation of SNORA31, CCL3L1, SNHG12, FTH1, MIR206, SLC39A7, CD9, or a combination thereof; downregulation of CHD4, SOX17, SNORA30, MIR126, MIR597, or a combination; or a combination of the expression patterns, compared to non-mass lesion type.

13. The method of claim 1, wherein if the breast cancer subtype of the patient is not triple-negative breast cancer and the MRI phenotype in step (a) is a mass lesion type, then gene information in step (b) is predicted to exhibit the expression pattern such that SNORA31, CCL3L1, SNORA71B or a combination thereof will be upregulated compared to non-mass lesion types.

14. The method of claim 1, wherein if the breast cancer subtype of the patient is triple-negative breast cancer and the MRI phenotype in step (a) shows increased standard deviation in Pre-T1 at SSF 5, then the gene information in step (b) is predicted to exhibit the expression pattern of: upregulation of CLEC3A, SRGN, DACT1, CGA, HSPG2, ABCC5, KMT2D, FBP1, VMP1, FZD2, or a combination thereof;
downregulation of PRDX4, NOP10, IGLC2, SNORA50, or a combination thereof; or a combination of the expression patterns.

15. The method of claim 1, wherein if the breast cancer subtype of the patient is HER2 positive and the MRI phenotype in step (a) shows increased postT1_mpp at SSF 2, then the gene information in step (b) is predicted to exhibit the expression pattern such that genes MLKL, POTEM, or a combination thereof will be upregulated.

16. The method of claim 1, wherein if the breast cancer subtype of the patient is HER2 positive and the MRI phenotype in step (a) shows decreased T2_mpp at SSF 5, then the gene information in step (b) is predicted to exhibit the expression pattern such that CXCL10 gene will be upregulated.

17. A method for treating breast cancer, the method comprising the steps of:
(a) identifying an MRI phenotype from MRI images obtained from a breast cancer patient;
(b) predicting one or more gene information differentially expressed according to the identified MRI phenotype and pathological molecular subtype;
(c) determining a personalized breast cancer treatment method from the predicted gene information; and
(d) treating the breast cancer patient with the treatment method determined in step (c).

18. The method of claim 17, wherein the method is selected from anti-estrogen therapy, adjuvant chemotherapy, prophylactic mastectomy, or a combination thereof.

19. A breast cancer treatment method selection system comprising the following components:
(a) a database where to search and extract information about genes related to breast cancer treatment;
(b) a communication unit capable of accessing the database;
(c) a first decision module for determining a tumor phenotype, using MRI images obtained from the patient;
(d) a second decision module for determining one or more gene information related to breast cancer by using the tumor phenotype;
(e) a third decision module for determining a treatment method for breast cancer from the derived gene information; and
(f) a display for showing the decision values determined by at least one of the decision modules.
